(19) United States
(12) Patent Application Publication
(10) Pub. No.: US 2026/0144581 A1
(43) Pub. Date: May 28, 2026

(54) IMPLANT OSSEUX EXPANSIBLE DE CHIRURGIE ORTHOPÉDIQUE HUMAINE, SYSTÈME ORTHOPÉDIQUE ET PROCÉDÉ DE FABRICATION DE L'IMPLANT

(71) Applicant: Lock-In VCF SA, Rolle (CH)

(72) Inventor: Guillaume LACAZE, La Rippe (CH)

(73) Assignee: Lock-In VCF SA, Rolle (CH)

(21) Appl. No.: 19/340,541

(22) Filed: Sep. 25, 2025

(30) Foreign Application Priority Data
Sep. 25, 2024 (EP) 24202388.5

(51) Int. Cl. A61B 17/88

(52) U.S. Cl. CPC A61B 17/8833

(57) ABSTRACT

An implant, a system, and a manufacturing process for human orthopedic surgery for restoring the volume and/or geometry of a bone by expanding at least two faces of the implant, each including a flange, the bringing together of the distal (12) and proximal (11) ends causing the support arms to pivot, causing the flanges to move away from each other and, consequently, the implant to expand, wherein an expansion sleeve or ring (20) and the proximal end of a central axis (3) of the implant can be moved away from each other to exert traction on the flanges, via expansion arms, which causes the support arms to pivot, resulting in controlled expansion of the implant (1) between the folded configuration and the deployed configuration.

The present application relates to the field of surgery, in particular human orthopaedic surgery and in particular to the treatment of a collapsed bone structure by restoring the volume of (or correcting) this bone structure. The present application relates in particular to an implant and to the method for manufacturing it, as well as to a system for restoring bone structure, in particular in the spine for the treatment (often called “reduction”) of compression fractures, in particular vertebral compression fractures (VCFs).

In this field, the problem of restoring the volume of bone structure that has collapsed is well known and the literature contains an abundance of solutions using expandable implants capable of passing from a folded configuration to a deployed configuration to restore the height of the bone structure, preferably in combination with an injection of bone substitute cement, known as cement (or bone cement). Many cements are known and they all have the advantage of being injectable in a liquid or viscous state for a certain period of time, then of hardening (by polymerization) inside the bone structure in order to stabilize it.

A major problem in this field concerns the expansion of the implant to restore height to the damaged bone tissue. Numerous solutions are known from the prior art, such as in particular from patent applications EP3086729, U.S. Pat. No. 11,540,926, EP3747385, EP2572680, EP3958752, EP2693967, EP2405835, U.S. Pat. No. 9,579,130, EP4216836, WO2023122005, WO2022162418, EP3843668 or U.S. Pat. No. 10,945,861 but these solutions present various problems of difficulty of handling for deploying to the expanded state, and of stability and reliability once deployed. Moreover, these known solutions are generally accompanied by an injection of bone cement, but do not provide any teaching relating to cement leakage outside of the implant, whereas such leakage can be detrimental to the surrounding tissue, or even to the entire organism if the chemical substances in the cement get into the bloodstream. In fact, cement generally comprises one or more polymerizable chemical substances, for example such as poly(methyl methacrylate) (PMMA) and possibly additives. In addition, the temperature reached during the polymerization of the cement is not harmless since it is generally greater than 60°.

Devices for the correction and stabilization (or bone fracture reduction), particularly of the spine in the form of a stent, or in the form of porous inflatable bags or of balloons, as in documents EP1408888 or EP1379185, possibly equipped with support flanges as in document US20060100706, are known from the prior art, in particular from documents EP1308134, U.S. Pat. Nos. 9,510,877, 8,936,627 or EP2467099. Numerous documents propose this type of stent, that is to say a deformable endoprosthesis similar to vascular stents, extenders or endoprostheses, which are generally in the form of a meshed tubular body, usually metallic and deformable by the introduction of an inflatable balloon to expand the body by spreading the meshes apart, the balloon then being removed to allow the injection of cement, which hardens and thus forms a correcting and stabilizing structure. However, these devices have the drawback of requiring implantation in two stages: the inflation of the balloon and then the injection of cement, which slows down and complicates the operation and also presents a risk of the device collapsing between the deflation of the balloon and filling of the stent with cement. Furthermore, these solutions have the drawback of not addressing the major problem of cement leaks.

Solutions using implants with a mesh structure, made of shape-memory metal, which is constrained into a folded shape for insertion into the bone tissue and capable of expanding spontaneously, when the stress is released and/or under heat, are also known, in particular from documents EP1938765, EP2351539 or WO200434924. These solutions have the drawback of using expensive alloys and complex manufacturing to obtain an adequate shape memory suitable for the intended implantation, which means additional cost when multiplied by the number of different implants necessary to cover the various pathological cases, in particular by the amplitude of the deformation of which the shape memory material is capable. Moreover, the force exerted by the return of the metal to its unstressed form is often not sufficient to properly correct the bone structure which has collapsed, or is at least a limiting factor. Furthermore, these solutions also have the drawback of not addressing the major problem of cement leaks.

Solutions using expandable implants that can bedeployed using a lever mechanism, in the manner of a car jack, to restore the bone structure to a determined height are also known from the prior art, in particular from documents EP2405835, U.S. Pat. No. 9,579,130, EP2572680 or EP1956990. These solutions have the advantage of not risking collapse unlike a stent that is deployed by a balloon which is removed before the injection of the cement, but also have the drawback of implantation in two stages and of the fact that the dimensions of the support surface for exerting the expansion force on the bone tissue are limited, in comparison with stents in particular. Furthermore, they also have the drawback of being expensive and of likewise not addressing the major problem of cement leaks.

The problem of cement leakage has already been identified, in particular in documents EP1408888, EP1509175 or WO200394805, which express the advantages that would be offered by a deformable implant that is not very permeable or that is impermeable, so as to limit or prevent cement leakage. These documents envisage numerous solutions for an expandable implant, made of metal or polymer, which could be either soft and flexible such as a membrane or a fabric, or even elastic, or semi-rigid (“conformable”) or rigid, or made of a shape memory material, with a continuous or fenestrated wall (i.e., mesh) and which could be porous or non-porous. However, all these hypotheses described in these documents define, above all, conceivable treatment methods and objectives to be achieved, without providing any real teaching as regards the technical characteristics or the structural arrangement of the implants, or on how to obtain such implants and thus implement these methods. These proposals therefore present a major problem of technical feasibility.

Furthermore, one problem which is not identified in the prior art concerns the cement injection site and the distribution of the forces exerted on the bone tissues to rectify them. Indeed, the impermeability of an implant makes it possible to avoid cement leaks, but the nature of the impermeable membrane and its technical characteristics such as its physico-chemical and mechanical properties influence its ability to deploy without breaking and to rectify the bone structure. Thus, an elastic membrane has the drawback of deforming excessively in low density zones and thus of having a limited capacity to restore height, with, in addition, the risk of breaking at the points at which its maximum elasticity is exceeded because of this uncontrolled deformation. A semi-rigid membrane is therefore preferable, but this problem of deformation also involves a problem of shapes of the implant, in the folded configuration and especially in the deployed configuration. Indeed, the shape of the deployed implant delimits the cement injection site and the control of this site is important for the distribution of the forces leading to the filling of the low density zones while rectifying the structure (especially in terms of height), and this has an impact on the success of the operation. Thus, it will be understood that the provision of an implant that addresses all of these problems is accompanied by a problem regarding technical feasibility and therefore manufacture.

Other recurring problems in orthopaedic surgery include invasiveness (i.e., the goal of making the smallest possible incision and of minimizing lesions) and also the deployment ratio in order to obtain a deployed implant that fills the largest possible volume while having been introduced through the smallest possible passage. Furthermore, this deployment ratio will have an impact on the distribution of forces for rectifying the vertebrae: if the deformability is too great, the cement-injection pressure will deform the pouch rather than restoring height.

A problem complementary to that of deployment concerns folding, which is generally not possible in implants of the prior art. Control of the folding allows control over the deployment and therefore over the injection site with a uniform distribution of the cement and of the pressure to fill the space to be filled following the collapse. Perfect proportionality suited to the fracture while at the same time respecting the shape of the bone inside the fracture is thus achieved.

In this context, it will be understood that there is still in this field a technical problem concerning the restoration of bone structure (rectification or reduction of fracture or increase in volume after collapse) by means of an expandable (deployable) implant that is able to expand collapsed bone tissue and is impermeable enough to prevent or limit the leakage of cement out of the implant with control of the injection site.

Finally, a main problem that still persists in the field concerns the technical feasibility of manufacturing implants proposed in the prior art. Document WO200394805, for example, describes numerous methods of administering substances and in particular bone cement, with numerous variants envisaged for an expandable implant, made of metal or polymer, which could be either soft and flexible such as a membrane or a fabric, or semi-conformable or rigid, or made of a shape memory material, with a continuous or fenestrated (i.e., meshed) wall and which could be porous or non-porous. However, that document describes only conceivable methods of treatment, but does not provide any teaching as to the technical features, or the structural arrangement of these many hypothetical implants used for these envisaged methods, or on how to obtain such implants and thus actually implement these methods. These proposals therefore present a major problem of technical feasibility and define objectives to be achieved rather than means for achieving them. Moreover, although many objectives have been detailed in the literature, many implants proposed for achieving these objectives have never seen the light of day due to problems of manufacture. In order to address the problem of manufacture, it is necessary to take into account the problems related to the desire to compact/fold a “bag” (balloon/pouch) made of a rigid and impermeable material in order to:
pass through a cylindrical conduit;
allow the pouch to expand without rupturing, despite the rigidity and the desired difference in volume between the folded volume and the deployed volume;
control the volume and the distribution of the expansion forces on the bone.

In this context, one object of the present invention is to overcome at least certain drawbacks of the prior art by proposing an implant for restoring a collapsed bone structure that is reliable and simple to handle and to implant.

This goal is achieved by an expandable bone implant for human orthopedic surgery for restoring the volume and/or the geometry of a bone, by expansion between a folded configuration and a deployed configuration, said implant comprising a central shaft and extending along a longitudinal axis between a proximal end connectable to an implantation instrument for holding the implant and a distal end intended to be inserted first into the bone, at least two faces, for example upper and lower, of the implant each comprising at least one flange for contact with bone tissue, each of the plates being supported by at least two support arms each, by means of a hinge on the central axis and a hinge under the respective flange of each of said support arms;

characterized in that:
an expansion sleeve or ring arranged in the same axis as said central axis;
at least two expansion arms, each comprising a hinge connecting them to one of the ends of one of the flanges and a hinge connecting them to said expansion sleeve or ring;
said expansion sleeve or ring and the proximal end of the central axis can be moved away from each other to exert traction on the flanges via the expansion arms, causing said support arms to pivot, thereby moving the flanges away from the central axis, so as to result in expansion of the flanges. expansion arms, which causes the support arms to pivot, causing the flanges to move away from the central axis, resulting in controlled expansion of the implant between the folded configuration and the deployed configuration.

According to another feature, it is the expansion ring that is operable by being able to cooperate, by means of hooks, with a hollow tube for gripping the implant of the implanting instrument, in which an expansion rod passes through the ring, while the central axis is able to cooperate with the expansion rod of said instrument, so that a thrust exerted on said expansion rod sliding inside said hollow tube causes said central axis to move away from said ring, resulting in an expansion of the implant controlled according to the thrust force exerted on the expansion rod.

According to another feature, it is the proximal end of the central axis that is actionable by being able to cooperate, by means of attachment, with the hollow tube for gripping the implant of the implantation instrument, while the expansion sleeve or ring is able to cooperate with the expansion rod of the instrument, so that a thrust exerted on said expansion rod sliding inside said hollow tube causes said expansion sleeve or ring to move away from said central axis, resulting in controlled expansion of the implant depending on the thrust force exerted on the expansion rod.

According to another feature, the central shaft comprises a conduit capable of cooperating with the expansion rod, which is hollow and provided with at least one opening at its distal end and can be connected to a fluid injection device to convey at least one fluid to the implant implantation site by means of the central axis. connectable to a fluid injection instrument to convey at least one fluid to the implant site via the central axis, preferably provided with openings to allow the fluid to flow out along the length of the central axis.

According to another feature, the support arms of the flanges comprise two arms connected respectively near the proximal and distal ends of their respective flanges, to provide support along the entire length of the flanges and limit the risk of bending or creep.

According to another feature, at least one additional central support arm is connected between a central portion of the central axis and a central portion of the flanges, with hinges at both ends of the support arm for pivoting.

According to another feature, the implant comprises two flanges arranged on either side of the central axis to provide support against damaged bone tissue on either side of the implant, for example to restore height or width.

According to another feature, the implant comprises at least one additional flange, the distribution of the flanges around the central axis varying according to their number and/or the requirements in terms of surgical treatment, preferably with an even angular distribution radially with respect to the central axis, in order to exert uniform compression on the bone tissue at the periphery of the implant.

Another distinctive feature is that the flanges have irregularities on their surface that comes into contact with the bone tissue, such as protrusions or, conversely, invaginations, to improve the grip of the flanges on the bone tissue. Such grip can be achieved by means of notches, ribs, grooves, straight or curved blades or chevrons, but also spikes, etc.

Another purpose of the present application is to overcome at least some of the disadvantages of the prior art by proposing a surgical intervention system that is easy to use and allows effective stabilization of bone tissue.

This purpose is achieved by an orthopedic treatment system for damaged bone tissue comprising at least one implant, a bone substitute cement, and at least one instrument for implanting and injecting cement into the implant, characterized in that said implant is an implant according to one of the embodiments described in this application.

According to another feature, the instrument for implanting and injecting cement comprises means for controlling the pressure and/or suction of the cement in order to fold the implant into a folded configuration if necessary.

According to another feature, the implantation instrument is separate but complementary to the injection instrument, whose cement injection channel passes through a channel inside the shaft of the implantation instrument holding the proximal end of the implant by means of its distal end.

Other features and advantages of the present invention will become more clearly apparent on reading the following description of various embodiments, with reference to the appended drawings, in which:

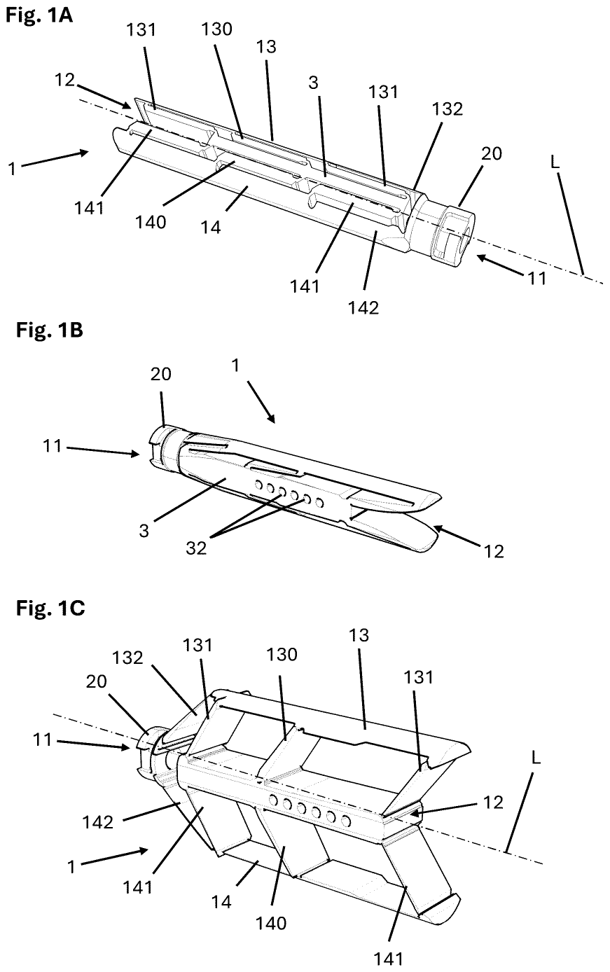

FIG. 1A depicts a perspective view of an expandable implant according to certain embodiments, FIG. 1B depicts a perspective view of an expandable implant according to another embodiment in the folded configuration, and FIG. 1C depicts a perspective view of the implant of FIG. 1B in the deployed configuration;

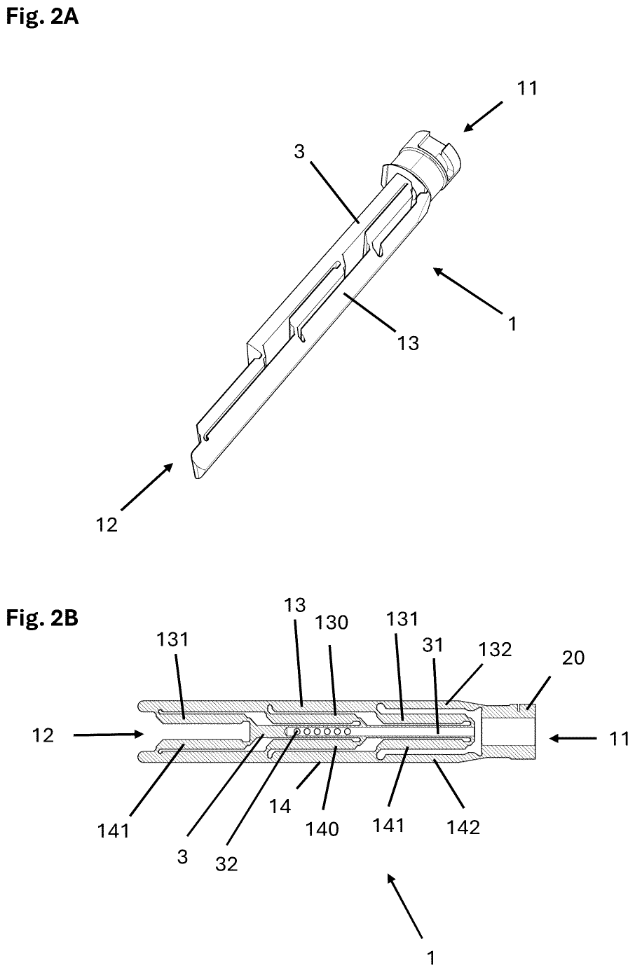

FIG. 2A depicts a perspective view of an expandable implant in the folded configuration according to certain embodiments and FIG. 2B depicts a cross-sectional view of the implant of FIG. 1B;

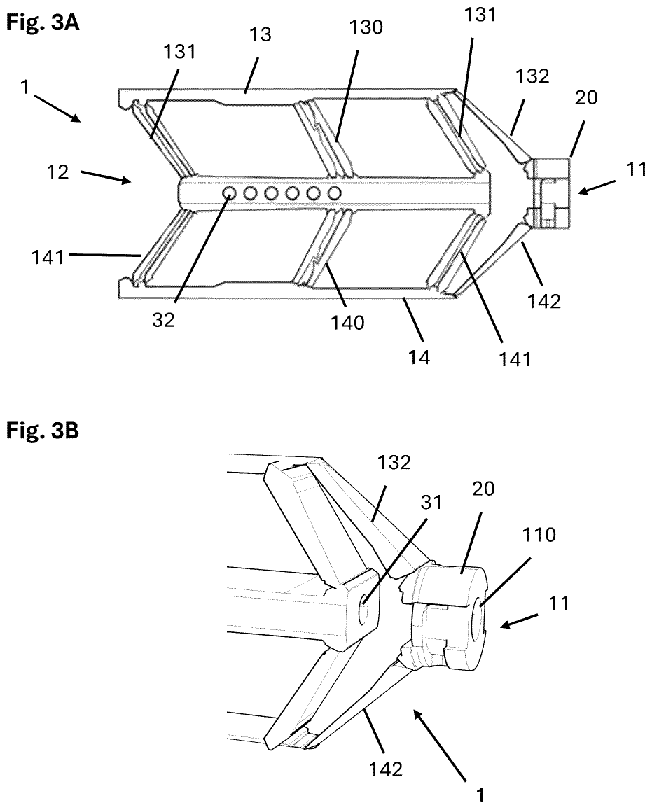

FIG. 3A depicts a profile view of an expandable implant in the deployed configuration and FIG. 3B depicts a detail of the proximal end of an expandable implant according to certain embodiments;

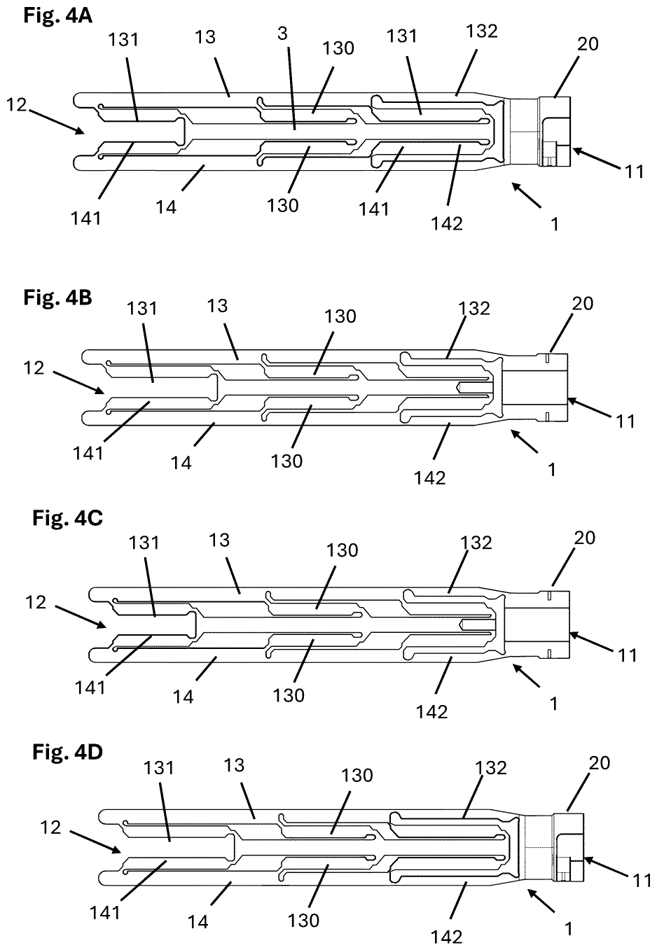

FIGS. 4A, 4B, 4C and 4D depict profile views of 4 expandable implants in the folded configuration according to different embodiments, with support arms of different lengths;

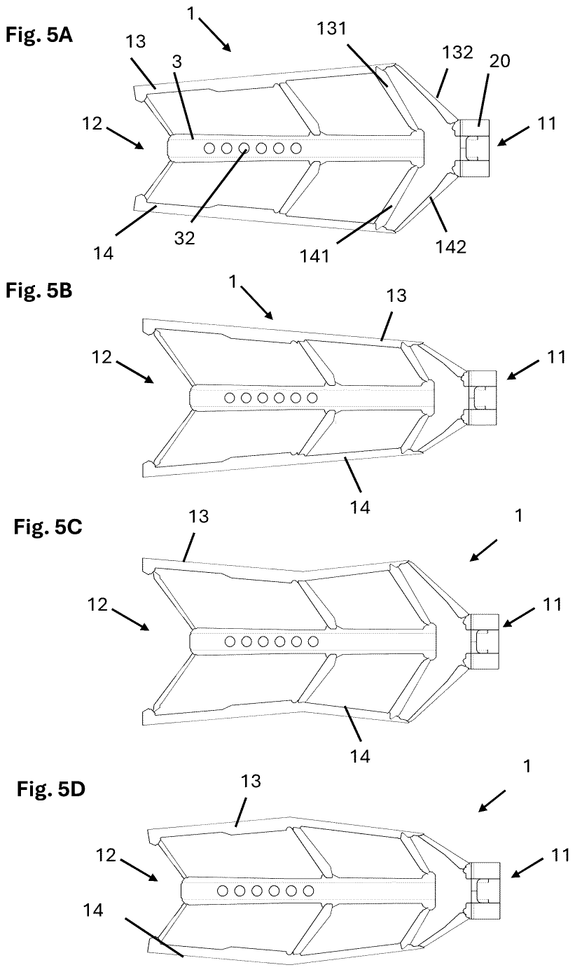

FIGS. 5A, 5B, 5C and 5D depict profile views of the implants of FIGS. 4A, 4B, 4C and 4D, respectively, but in the deployed configuration with their flanges non-parallel;

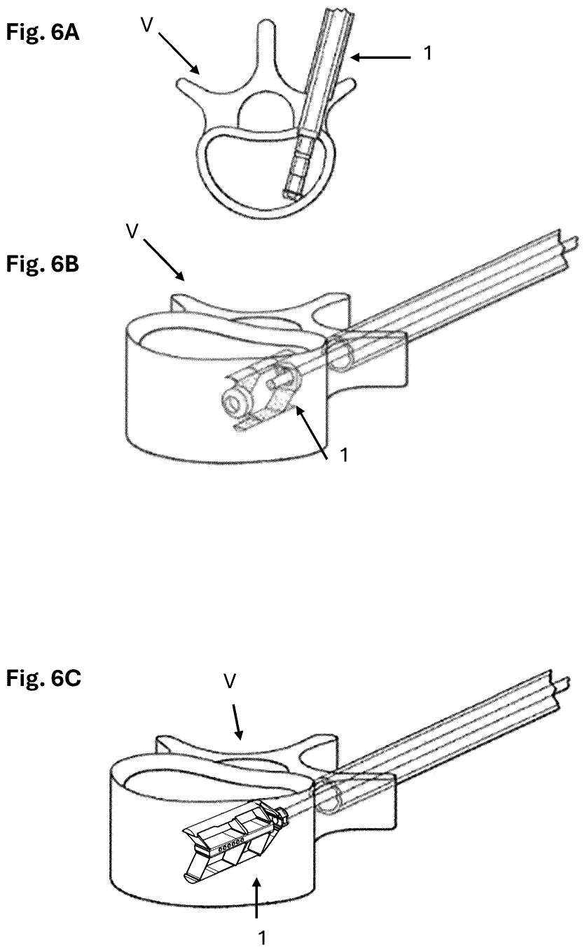

FIG. 6A depicts a view from above of a vertebra in which an implant according to various embodiments is implanted;

FIG. 6B depicts a perspective view of a vertebra in which an implant of the prior art is implanted;

FIG. 6C depicts a perspective view of a vertebra in which an implant according to certain embodiments is implanted;

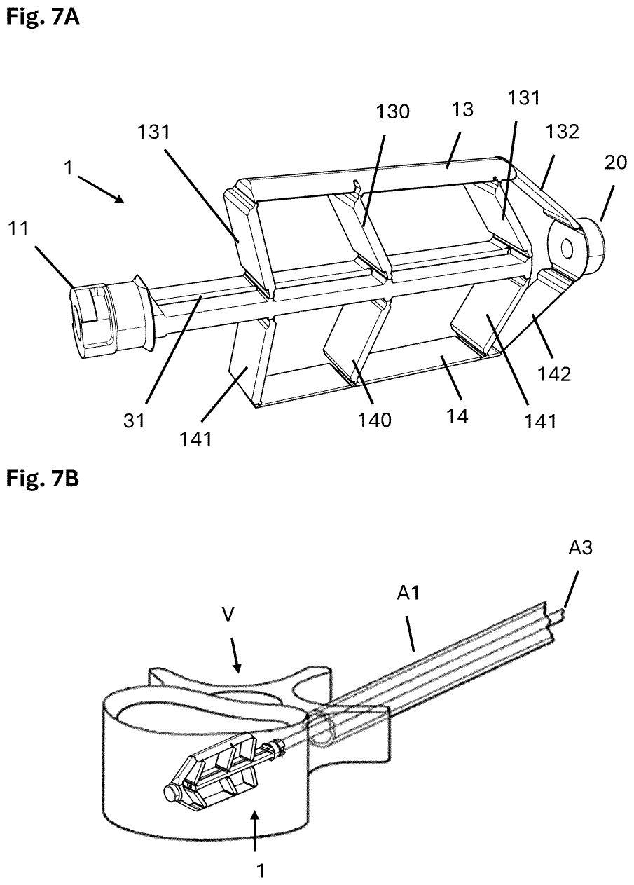

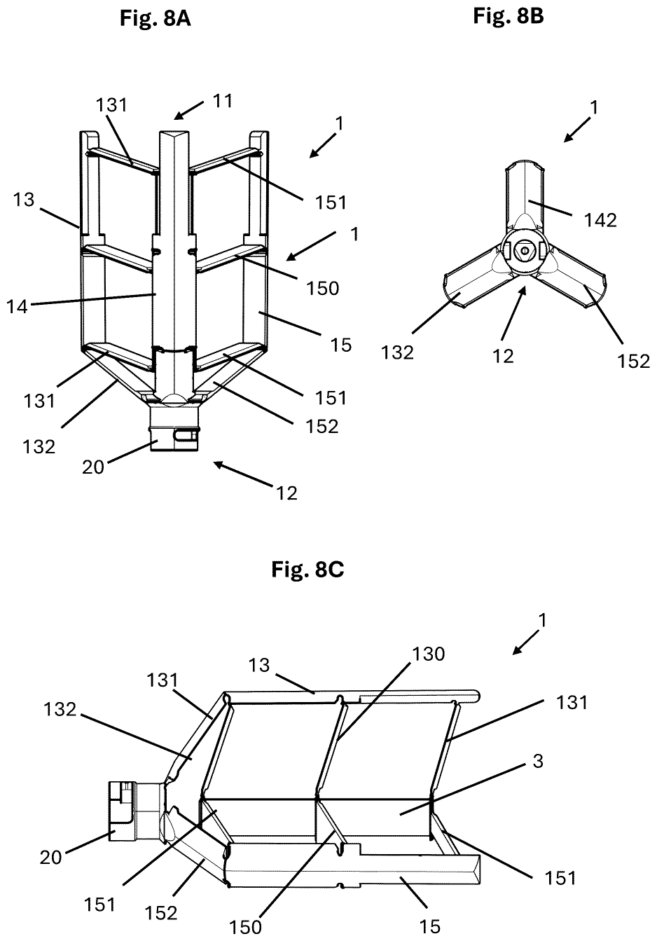

FIG. 7A depicts a perspective view of an expandable implant according to one of certain embodiments, which is inverted, and FIG. 7B depicts a perspective view of a vertebra in which the implant of FIG. 7A is implanted, using an instrument FIGS. 8A, 8B and 8C depict, respectively, a view from above, a face-on view of the profile view of an expandable implant with three flanges according to certain embodiments;

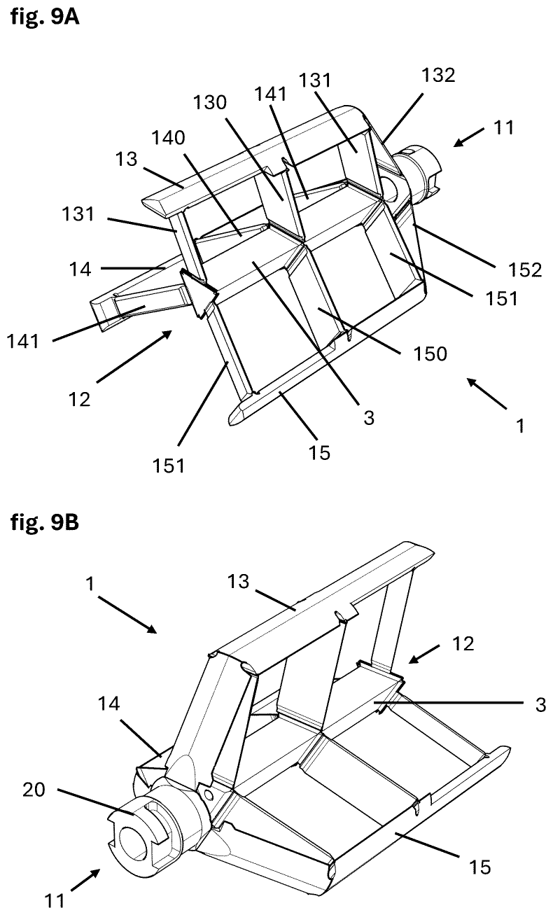

FIGS. 9A and 9B depict perspective views respectively from the rear and from the front of an expandable implant according to the embodiments of FIGS. 8A, 8B and 8C;

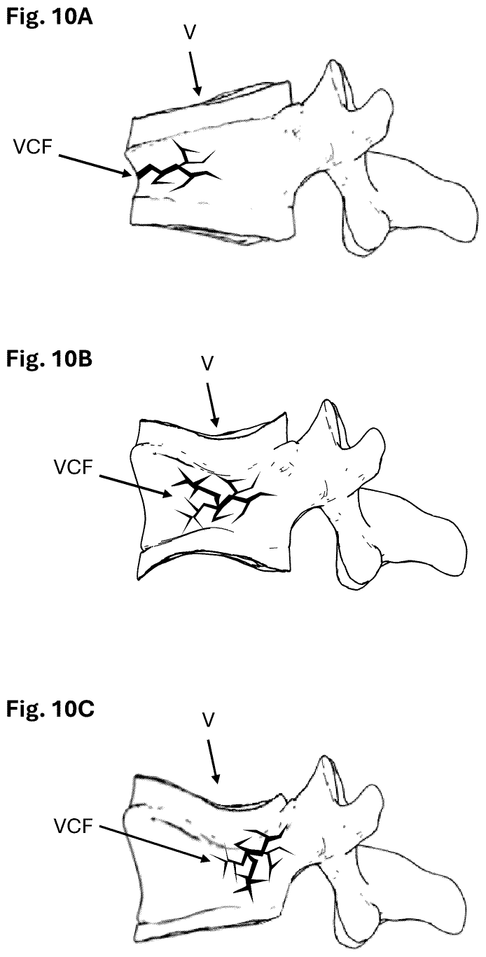

FIGS. 10A, 10B and 10C depict profile views of vertebrae that have respectively suffered anterior, median and posterior vertebral compression fractures (VCFs);

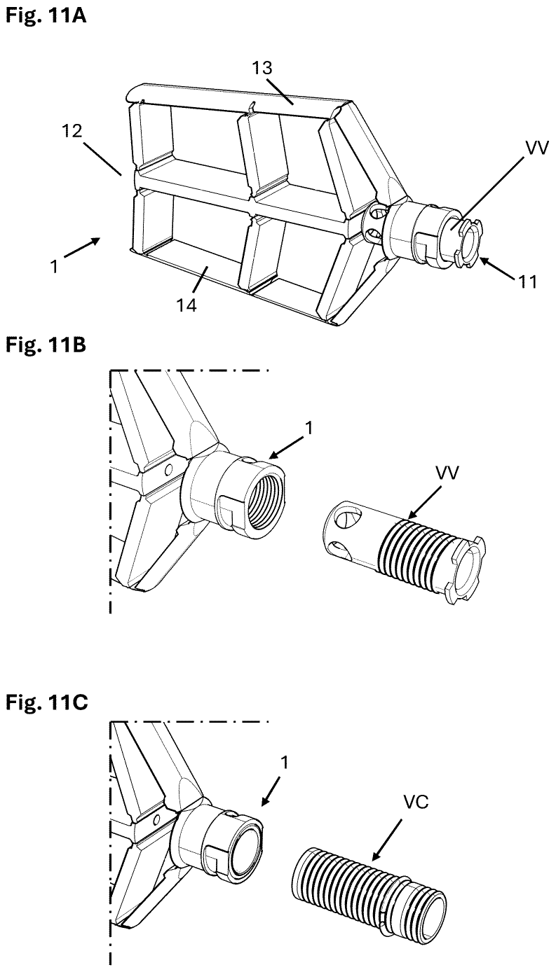

FIG. 11A depicts a perspective view of an expandable implant in the deployed configuration and equipped with a lock that holds the implant in its deployed configuration, FIG. 11B depicts a detail of the proximal end of an implant of the type of FIG. 11A, with the lock on the outside of the implant according to certain embodiments, and FIG. 11C depicts a detail of the proximal end of an implant according to certain embodiments with another type of lock on the outside of the implant;

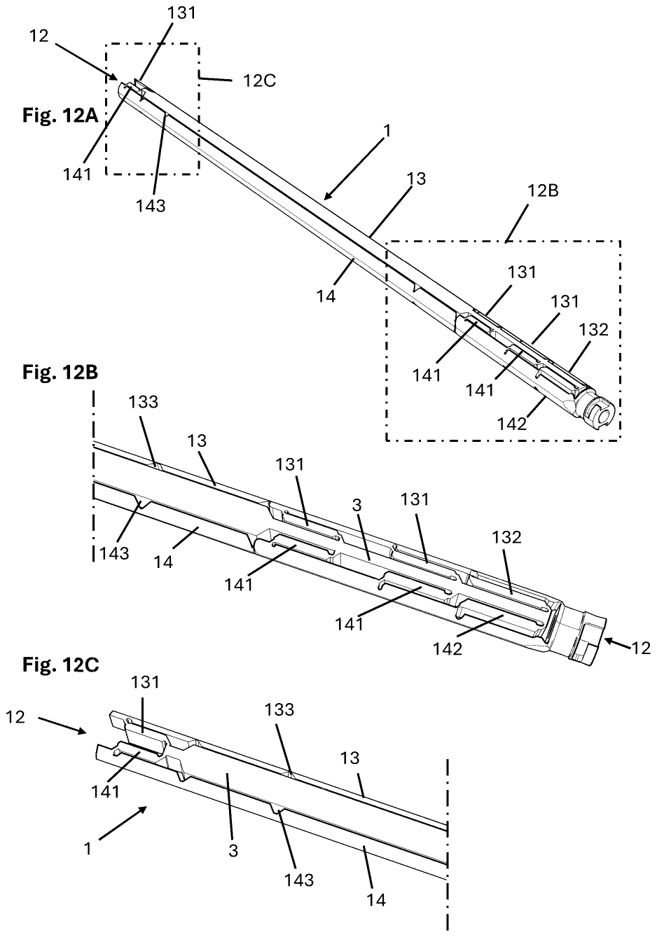

FIG. 12A depicts a perspective view of an implant according to certain embodiments and FIGS. 12B and 12C depict, respectively, the detail of the boxes 12B and 12C of FIG. 12A of the implant.

The present application relates to an implant and an orthopaedic surgery system for treating fractures in bone and bone tissue in general, and to a method for manufacturing the implant. The bone implant is preferably a spinal implant, and in particular a vertebral or even in fact intervertebral implant, although other uses elsewhere than in the spine (intervertebral discs) or in other bony structures where it is necessary to fill a space left vacant as the result of a fracture (the causes of which may be various, even though they generally imply a reduction in bone density) are conceivable. Thus, vertebral compression fractures (VCFs) are a favourite application but are not the only conditions that can be treated using the present invention, and the person skilled in the art will appreciate the possibilities offered without requiring further details here. In terms of other bones, mention may be made of the femur or the humerus (head), for example in the event of a risk of collapse, and the implant is able to be implanted directly in the medullary cavity. In addition, the tibial plateau is frequently subject to crushing, and the implants or systems of the present application are useful for restoring height in any type of bone crushing or collapse, for example in the distal part of the humerus or femur. On the other hand, as taught, for example, in document EP2921142, it is possible to use expandable implants as bone anchoring implants, and such use is also possible for implants such as those of the present application. In this case, the implants will be extended at their proximal end by an elongated body to which another orthopedic implant of another type or a surgical device for fixing other elements can be attached. Nevertheless, in the case of use as a bone anchor in a vascularized structure, such as a humeral or femoral head, the size of the implant will preferably be limited in relation to the bone structure in order to preserve vascularization and promote bone healing. Certain embodiments comprising more than two flanges may notably prove more effective in the treatment of long bones of this type by distributing the forces of expansion over more than two surfaces, thereby providing better stability regardless of the type of bone. However, the use of the implant in long bones is not limited to the embodiments with more than two flanges because, depending on the type of fracture, an implant with two flanges may still be suitable for use in long bones, as for example depicted in FIGS. 12A, 12B and 12C. The only notable features for the treatment of long bones are the length of the flanges and especially the length of the support arms, which generally varies according to the position inside the medullary cavity. In particular, the medullary cavity is generally wider at the ends of the bone than at the centre, and various embodiments make it possible to conform to this shape of the medullary cavity, by virtue of the presence of support arms (131, 141) of different lengths in order to obtain a greater or lesser expansion at different points of the implant, as for example depicted in FIGS. 12B and 12C. Thus, with longer support arms (131, 141) at the ends, the expansion of the implant will be greater at the ends of the implant, which will therefore be disposed at the epiphyses of the long bone, for example. It will be noted that in this kind of case, it is possible to provide an articulation of the flanges themselves in the zones in which the expansion is variable. Such an articulation may naturally consist in thinning the flanges at the appropriate points, according to the lengths of the support arms, for example as depicted in FIGS. 12B and 12C.

Likewise, in various embodiments that do not necessarily relate to long bones, the implant comprises distal-end support arms that have a length different from that of the proximal-end support arms and/or of the central support arms, so that the implant in the deployed configuration has flanges that are not parallel to one another. Illustrative and nonlimiting examples of such embodiments are depicted in FIGS. 4A, 4B, 4C and 4D in the folded configuration and in FIGS. 5A, 5B, 5C and 5D corresponding to their respective deployed configurations. It will be appreciated that longer arms at the proximal end (FIGS. 4A and 5A) make it possible to obtain flanges that are inclined towards the distal end, whereas longer arms at the distal end (FIGS. 4B and 5B) make it possible to obtain flanges that are inclined towards the proximal end. In addition, it is possible to provide flanges that do not remain flat at the end of expansion, this representing a marked advantage of the implant which is then able to conform to the anatomical shapes of the bones in which they are implanted. Thus, a concave or bi-concave shape (concave on both faces of the flanges) of the deployed implant can be obtained with central arms (130, 140) that are shorter than the support arms (131, 141), as in FIGS. 4C and 5C, whereas a convex (or bi-convex shape which is convex on both faces of the flanges), can be obtained with central arms (130, 140) that are longer than the support arms (131, 141), as in FIGS. 4D and 5D.

Certain embodiments provide for the injection of a fluid (e.g. “bone cement”, generally based on a polymer such as PMMA for example and well known to those skilled in the art such that no detail on the cement will be given here). Thus, once positioned, the implant may notably be stabilized by such an injection of cement. However, because cement leaks remain a problem, it is preferable not to use cement with the implant described here if the cement is not contained, at least in instances in which the fracture is so large that the cement can leak into the surrounding tissue.

The term “secured” here means the two elements are secured to one another, either permanently (or near-permanently) but also sometimes that a connection is made so that one element can be actuated by another. Thus, screw-fastening or collaboration between shapes for temporarily locking the elements together are covered by this nonlimiting term.

The terms “ring”, “sleeve”, or “tube” refer to hollow structures such as bands, conduits or pipes, but nonlimitingly, notably having various shapes (on the inside as on the outside), although a cylindrical shape is preferred. The term “canal” by contrast is preferably used here to refer to a passage rather than to the element that contains it, and the term “opening” here refers to the fact that an element is open and able to be passed through, opening out into another structure or another element. In general, the terms “sleeve”, “tube” or “conduit” refer to longer elements than rings or bands, although their use here is likewise nonlimiting. Furthermore, the terms “socket” or “cup” refer also to hollow structures that are open at one end but closed at the other end, such as plugs, closures, constrictions or restrictions, and these terms are used indiscriminately without any limitation.

The term “hinge” is used here in its functional sense without implying any structural limitation, and may in fact refer to mechanical hinges even though these are preferably formed (as illustrated in the nonlimiting examples in the figures) by thinning (or narrowing, removing material from) elements such as the support arms or other elements. Thus, a hinge is in fact an articulation point or region since it is known in the art that there is generally no danger associated with providing such pivot mechanisms in implants because the materials of which they are made are suited to this type of articulation. It will be noted that thinnings of the flanges are also conceivable, notably next to the articulations (hinges) of the support arms, so as to allow the flange to conform to the desired morphological shape, notably when the support arms do not have the same length as each other.

Moreover, the present invention makes it possible to control the shape of the implant once deployed, for example as illustrated in FIGS. 4A, 4B, 4C, 4D, 5A, 5B, 5C and 5D. A fluid-injection instrument (Ac) may be provided to fill the implant with a fluid such as bone cement. Such an instrument may be fitted with means for controlling the injected pressure (a pressure gauge for example) and for indicating the resulting volume so that the expansion into the bone tissue can be controlled effectively.

Finally, it will be appreciated that the instrumentation proposed in the present application in certain embodiments, using an implant holder (or ancillary) of relatively conventional type to hold the implant and introduce it into the bony tissue, but also of less-conventional type for expanding it into the bony tissue, also offers the advantage that all the implantation and stabilization steps can be carried out using just one single instrument and in a continuous operation. Specifically, the ancillary with a hollow tube for conveying cement through the tube that holds the cement makes it possible to offer an instrument that allows the surgical intervention to be performed quickly and efficiently. After drilling, the implant is introduced and, without withdrawing the instrument, cement can be injected and then the tool can be withdrawn before, during or even after the polymerization of the cement (for example using a mechanism for cutting the hardened cement as the instrument rotates). The time taken to perform the surgical operation is of course markedly reduced as also the stability of the implant which is not released at any time until it has been stabilized by the injection of cement filling all the free volumes around it, unlike in certain solutions of the prior art.

In general, the present application relates to an expandable bone implant (1) for human orthopaedic surgery for restoring the volume and/or geometry of a bone by expansion between a folded configuration and a deployed configuration, said implant comprising a central shaft (3) and extending along a longitudinal axis (L) between a proximal end (11) adapted to cooperate with an implantation instrument (A) for holding the implant and a distal end (12) intended to be first inserted into the bone, at least two faces, for example, an upper and a lower face, of the implant each comprising at least one flange (13, 14, 15) for contact with bone tissue, each of the flanges being supported by at least two support arms (131, 141, 151) each, by means of a hinge on the central shaft (3) and a hinge under the respective flange (13, 14, 15) of each of said support arms (131, 141, 151); This proximal end can be connected to a gripping instrument (known as an implant holder) and is therefore capable of cooperating with the latter by means of attachment or physical connection, for example, as known to those skilled in the art. However, certain embodiments provide specific and advantageous attachment means to facilitate the gripping of the implant by an implant holder and, above all, the release of the implant by an L-shaped movement of the implant holder. On the other hand, by being connectable to the instrument, the implant is generally operable for expansion (in particular by injecting a fluid into it and/or by pushing or pulling on an element of the implant), as is widely known in the prior art. Indeed, many systems include expandable implants that can be actuated when mounted on an implant holder that includes an actuating means for expanding the implant (generally a conduit and/or a rod passing through the implant holder to open into a cavity in the implant and/or cooperate with an implant component that allows its expansion, the actuation generally involving a pushing and/or pulling force). The skilled person will therefore understand from reading this application that the implant can be defined without further detail on the instrument and the actuation, since these are conventional mechanisms in the field and the system comprising the implant and the instrument is of course fully defined, but thatthe implant alone is in fact also well defined in its operable nature independently of the instrument and without unnecessary detail on the actuation mechanism (sliding rod, for example) insofar as these are perfectly standard or conventional mechanisms in the field. It is understood in the field of the present application that the term “actuable” implies pushing or pulling (with or without rotation), and the present application thus provides sufficient explanation for the implant to be considered sufficiently clearly defined without additional reference to the instrument enabling its actuation. Conversely, certain embodiments may relate to the instrument itself, through the originality of its elements allowing the implant to be gripped and/or actuated for expansion, and these characteristics then define the instrument independently of the implant since they do not require any particular details about the implant other than those relating to the function performed by the instrument.

Such an implant (1) is preferably characterized in that:
an expansion ring or socket (20) is arranged on the same axis as said central shaft (3);
at least two expansion arms (132, 142, 152) each have a hinge connecting them to one of the ends of one of the flanges (13, 14, 15) and a hinge connecting them to said expansion ring or socket (20);
said expansion ring or socket (20) and the proximal end of the central shaft (3) are able to be actuated in order to cause separation between said socket (3) and said central shaft (3), applying traction to the flanges (13, 14, 15), by means of the expansion arms (132, 142, 152), thereby causing said support arms (131, 141, 151) to pivot, causing the flanges (13, 14, 15) to move away from the central shaft (3), so as to result in controlled expansion of the implant (1) between said folded configuration and said deployed configuration. Actuation of the ring or socket (20) and the central shaft (3) is preferably obtained through the fact that these are able to collaborate, respectively or vice versa, with a hollow tube (A1) used by an implantation instrument (A) for holding the implant (1), and with an expansion rod (A3) of said instrument (A), this expansion rod (A3) being able to slide inside said hollow tube (A1) to move said socket (20) and said central shaft (3) away from each other.

It will be appreciated that, unlike in certain implants of the prior art in which the structure used for expansion (often with a pull-shaft) that enables two ends of the implant to be brought closer together and the support arms to be brought closer together, the implants of the present application are deployed without bringing the support arms closer together, thereby avoiding the need to apply two mutually opposing forces to the same elements, since the force exerted on the expansion arms (132, 142, 152) enables the support arms (131, 141, 151) to be made to pivot in the same direction, thereby limiting the stresses applied to the structures and thus making deployment easier and also preserving these structures and therefore improving the reliability of the implant.

It will be noted that the implant comprises at least one flange and that it is therefore possible to have a single flange for expansion on just one side, for example as depicted in FIG. 2A, as known in the prior art on deployable implants comprising other deployment mechanisms such as those above and, on the contrary, for example with support arms that move closer together for the expansion of the implant.

It will be noted that the term “central reinforcement” is used to refer to a central shaft on which the deployment support arms are articulated to one another. The implant flanges to which the support arms are articulated are also articulated to this shaft, which therefore acts as a reinforcement but mainly as a support for expansion because it supports the articulations or hinges of one of the flanges. Furthermore, it will be noted that the expansion (i.e. support) arms are advantageously connected to the flange directly so that the force of expansion of the implant can be applied and the flanges parted directly by direct action on these flanges by applying a force in a single direction.

In certain embodiments, it is said expansion ring (20) that is able to collaborate with a hollow tube (A1) used by said implantation instrument (A) for holding the implant (1), letting the expansion rod (A3) pass through the ring, while the central shaft (3) is able to collaborate with said expansion rod (A3) of said instrument (A) so that a pressing force exerted on said expansion rod (A3) sliding inside said hollow tube (A1) causes said central shaft (3) to move away from said ring (20), resulting in controlled expansion of the implant (1) dependent on the pressing force exerted on the expansion rod (A3).

In certain embodiments that are alternatives to the preceding ones, it is the proximal end of the central shaft (3) that is actuable by being able to collaborate, by means of a coupling, with said hollow tube (A1) used by said implantation instrument (A) for holding the implant (1), whereas said expansion ring or socket (20) is able to collaborate with said expansion rod (A3) of said instrument (A) so that a pressing force exerted on said expansion rod (A3) sliding inside said hollow tube (A1) causes said expansion ring or socket (20) to move away from said central shaft (3), resulting in controlled expansion of the implant (1) dependent on the pressing force exerted on the expansion rod (A3).

In certain embodiments, the central shaft (3) comprises a conduit (31) able to collaborate with said expansion rod (A3) which is, on the one hand, hollow and provided with at least one opening at its distal end and, on the other hand, able to be connected to a fluid-injection instrument (AC) for conveying at least one fluid to the implant implantation site via the central shaft (3), and which is preferably provided with openings (32) to disgorge the fluid along the length of the central shaft (3).

In certain embodiments, the flange-support arms (131, 141, 151) comprise two arms connected respectively near the proximal and distal ends of their respective flange (13, 14, 15) to provide support along the entire length of the flanges and limit the risk of them bowing or sagging.

In certain embodiments, at least one additional central support arm (130, 140, 150) is connected between a central portion of the central shaft and a central portion of the flanges (13, 14, 15), with hinges at the two ends of the support arm (130, 140, 150) to allow it to pivot.

In certain embodiments, two flanges (13, 14) are arranged one on each side of the central shaft (3) to provide a bearing surface to bear against the damaged bone tissue on each side of the implant, for example to restore a height or a width.

In certain embodiments, the implant comprises at least one additional flange (15), the distribution of the flanges about the central shaft (3) varying according to the number of them and/or according to what is needed in terms of surgical intervention, these preferably being equally angularly distributed radially with respect to the central shaft so as to apply uniform compression to the bone tissue at the periphery of the implant. In certain embodiments, double support arms are provided to strengthen the structure. Furthermore, locking means are provided in certain instances, to prevent the implant from folding of its own accord. The very fine diameters of the implants and their central shafts are difficult to reconcile with screw threads for performing the expansion by a screwing action at the implant, although it is advantageous to perform a screwing action at the instrument actuating expansion, particularly when the expansion involves bringing the support arms closer to one another. Thus, as known from the prior art, it is possible for example to use a split ring housed in a circular recess of the implant and engaging with teeth on the push-or pull-shaft, which teeth are oriented in such a way as to allow this shaft to turn in only one direction, for example as depicted in FIG. 11C. Thus, the shaft may be actuated for expanding the flanges by rotating past successive teeth, thereby allowing the implant to be locked in the deployed configuration.

Unlike certain implants of the prior art in which the structure used for expansion (with, as often, a pull-shaft) that enables two ends of the implant to be brought closer together and the support arms to be brought closer together necessarily has to remain in place, the implants of the present application are deployed without any moving-together of the support arms, which advantageously allows these arms to be locked with a screw lock and means that such teeth that make the task difficult and offer reduced reliability are not needed. Thus, for example as depicted in FIGS. 11A and 11B, it is possible for example to use a screw threaded sleeve configured to be arranged in the hollow tube of the implantation instrument (A) holding the implant (and surrounding any fluid-injection conduit that may be present inside it) . Such a sleeve therefore has a screw thread intended to collaborate with a tapped thread belonging to the proximal end (11) of the implant and has actuating means for screwing or unscrewing (such as radial wings depicted in FIG. 11C) .

In addition, the configuration of the implants of the present application offers a not-insignificant advantage as far as the reliability of expansion is concerned. Specifically, the fact that support arms are positioned at least at the ends of the flanges (and possibly with one or more reinforcing arms between the ends) enables the creation of deformable parallelograms that maintain their property of mutual-parallelism of their sides, unlike certain implants of the prior art where the support arms are mounted in opposition. This type of implant of the prior art generally requires for the arms to be provided in duplicate on each side in order to improve the reliability of expansion. The implants of the present application do not require the arms to be duplicated, although it does remain possible to do so, particularly in the case of implants of a large size and/or intended to bear a significant load. Thus, certain embodiments comprise support arms in duplicate, at least at one of the positions of these arms, and preferably at each of them, for example as depicted in FIG. 3A. In addition, such double arms may have a self-locking mechanism that locks them in the deployed configuration, such as, for example, teeth formed facing one another so that they engage with one another, for example as depicted for the central support arms (130, 140) in FIG. 3A.

The present application also relates to a system for orthopaedic treatment of damaged bone tissue, comprising a bone substitute cement and at least one instrument (A) for implantation and for injection (Ac) of cement into the implant (1), characterized in that it comprises an implant (1) according to various embodiments.

In certain embodiments, the instrument (A) for implantation and for injection (Ac) of cement comprises means for controlling the pressure and/or the aspiration of the cement so that the implant can be re-folded to the folded configuration if necessary. This type of means is widely known and may, for example, simply be based on a piston actuated in a cylinder, but may comprise more complex means, as is widely known in the field of the present application.

In certain embodiments, the implantation instrument (A) is distinct from but complements the injection instrument (Ac) the cement-injection canal of which passes through a canal inside the rod of the implantation instrument (A) that via its distal end holds the proximal end of the implant (1).

The illustrative and nonlimiting figures of the present application will now be described in detail the better to explain the various embodiments and provide examples of structural elements that can be used in the foregoing context. That which follows must not be considered as being limiting since the various elements or components illustrated are merely examples and the figures may combine elements or components that are not necessarily dependent on one another.

FIG. 1A depicts a perspective view of an expandable implant according to certain embodiments, FIG. 1B depicts a perspective view of an expandable implant according to another embodiment in the folded configuration, and FIG. 1C depicts a perspective view of the implant of FIG. 1B in the deployed configuration. In FIG. 1A, it will be noted that the expansion ring comprises an L-shaped groove that allows the implant to be held by a tool comprising a holding means that is able to be actuated by a “quarter-turn” movement that complements the L-shape, thereby making assembly with the implant holder, and especially the release of the implant on completion of the operation, easier. The term quarter turn can be used herein to refer to a conventional mechanism, but it is obvious that such a means does not necessarily require a 90° rotation and that it can be more or less, although a significant rotation is preferable before the implant can be released. In FIG. 1A, it will be noted that the proximal end (11) comprises a conduit allowing cement to be injected into the implant and in FIG. 1B it will also be noted that the central shaft (3) comprises holes (32) provided so that the cement injected via the proximal end can reach the inside of the shaft (3) then provided with a central conduit (31) itself opening onto the holes (32), so as to make easier the distribution of the cement inside the implant and in particular the space cleared by virtue of the deployment of the implant by the parting of the flanges, for example as can be seen particularly in FIG. 2B. FIG. 2A depicts a perspective view of an expandable implant in the folded configuration according to certain embodiments and FIG. 2B depicts a cross-sectional view of the implant of FIG. 1B. FIG. 2A in fact depicts a variant that is similar to certain implants of the prior art having just one flange, but with the arm mechanism pivoting in the same direction which is a concept common to most of the embodiments detailed in the present application. This kind of mechanism with just one flange therefore allows deployment in just one given direction perpendicular to the longitudinal axis, which may be beneficial in certain cases. The present disclosure naturally allows for this kind of embodiment.

It will also be noted that the articulations between the central shaft and the flanges in the examples depicted and particularly for that of FIG. 2B differ according to the position of the arms with respect to the flange. Specifically, because of constraints associated with deployments achieved by the parting of the flanges, it is necessary in the case of flexible hinges as depicted in the figures for the indentation (removal of material) enabling articulation to be greater in size at the proximal end than at the distal end, because their respective axes of pivoting are different. It will also be noted that the hinges depicted in these figures of the present application are in fact flexible zones, thanks to these indentations or removals of material that confer flexibility on the material used, whether this be a material that is basically flexible (for example such as PEEK, polyether ether ketone) or one that is basically not (for example such as titanium alloys) . Such flexible hinges require greater removals of material than in the case of mechanical hinges where such a requirement to remove is no longer needed, or even where there is rather a need to thicken the material in order to provide the hinge pin of the mechanical hinge. Thus, these flexible hinges are preferred in most cases.

FIG. 3A depicts a profile view of an expandable implant in the deployed configuration and FIG. 3B depicts a detail of the proximal end of an expandable implant according to certain embodiments. In particular, FIG. 3A depicts one example of certain embodiments in which each of the support arms or at least some of the support arms (130, 140, 131, 141) are duplicated into pairs of adjacent arms and could be triplicated in other embodiments. In addition, some of the support arms (the central arms (130, 140) in this example) have projections that complement those of the other support arm of the same pair, so that in the deployed configuration the two projections come into abutment against one another so as to oppose the re-folding of the implant once a certain degree of expansion (separation) has been exceeded.

FIGS. 4A, 4B, 4C and 4D depict profile views of 4 expandable implants in the folded configuration according to different embodiments, with support arms of different lengths. Similarly, FIGS. 5A, 5B, 5C and 5D depict profile views of the implants of FIGS. 4A, 4B, 4C and 4D, respectively, but in the deployed configuration with their flanges nonparallel. In certain embodiments, the flanges are articulated to support arms (131, 141) the lengths of which differ from one flange to another in order to achieve asymmetric expansion. It will be appreciated that these FIGS. 4 and 5 represent nonlimiting examples of particularly advantageous embodiments in which the support arms have different lengths according to their position along the longitudinal axis. Thus, for example, in the case of FIGS. 4A and 5A, the flanges, once the implant has been deployed, are notably able to achieve kyphosis in the case of vertebral implantation, through the fact that the distance between the flanges at the distal end is shorter than the distance between the flanges at the proximal end, whereas FIGS. 4B and 5B depict examples in which the distance between the flanges at the distal end is greater than that at the proximal end. In addition, FIGS. 4C, 5C, 4D and 5D depict other particularly advantageous and unheard-of embodiments in which the flanges are able, during the course of their deployment, to adopt a shape different from that which they have in the folded position, thanks to their flexibility or to various notches distributed over at least one of their (internal or external) faces. For example, in FIGS. 4C and 5C, the flanges are concave in shape, which means to say that the distance(s) separating them at the distal and proximal ends is (are) greater than the distance separating them between these 2 ends. Conversely, in FIGS. 4D and 5D, the flanges once deployed are convex in shape, as a result of the fact that the distance(s) separating them at the proximal and distal ends is (are) less than the distance separating the flanges between these two ends. These various configurations are obtained through the fact that the support arms have length dimensions that differ from one arm to another along the longitudinal axis, and it is also possible for these lengths to differ between the arms of one flange compared to those of the arms of the other flange. In addition, in the embodiments in which the support arms have lengths that vary according to their position along the longitudinal axis, it is possible also to have different lengths from one flange to the other, for example in order to obtain a deployed implant in which one of the flanges is concave and the other convex. It will be appreciated that numerous combinations regarding the various flanges of the implant are thus possible (irrespective of the number of them) . For example, by considering the upper half of one of FIG. 4A to 4D or 5A to 5D, for one flange and the lower half of another of these figures, it will be appreciated that it is possible to have a large number of combinations each offering particular advantages depending on the geometry of the structure that is to be restored.

FIG. 6 depicts a view from above of a vertebra in which an implant according to various embodiments is implanted, FIG. 6B depicts a perspective view of a vertebra in which an implant of the prior art is implanted, whereas FIG. 6C depicts a perspective view of a vertebra in which an implant according to certain embodiments is implanted. It will be appreciated from these figures how the implant may be implanted in a vertebral body to treat a vertebral compression fracture and show that the implant can conform more reliably to the space it fills, thanks to flanges that are supported at their ends, but also thanks to the envelope which, once filled with cement, will completely solidify thedeployed implant and the bone structure.

FIG. 7A depicts a perspective view of an expandable implant according to one of certain embodiments, which is inverted, and FIG. 7B depicts a perspective view of a vertebra in which the implant of FIG. 7A is implanted, using an instrument. FIG. 7A depicts certain embodiments in which the extension of the implant is the reverse of that of the other embodiments, i.e. the expansion ring (20), instead of being positioned at the proximal end in order to apply traction to the flanges, is positioned at the distal end in order likewise to apply traction to the flanges but towards the distal end thanks to a pressing force transmitted through the implant and particularly along the central shaft. In these embodiments, the proximal end (11) therefore has passing through it a conduit through which an instrument is capable of passing as far as the expansion ring (20) at the distal end in order to apply a pushing force while the proximal end (11) is held fixed by an implant holder. The flanges therefore part as a result of the pivoting of the support arms (131, 141) with respect to the central shaft, in a movement oriented towards the distal end. These embodiments may be particularly advantageous particularly in instances in which it is preferable to limit the stresses on the bone tissue at the proximal end and limit the size of the implant at the proximal end. Specifically, what is thus obtained is an implant that deploys towards the distal end, there remaining at the proximal end only part of the central shaft (3) and the proximal end by which the implant was held by the implant holder. The flanges thus deploy towards the front, thereby limiting the risks of breakage at the proximal end for example as depicted in FIG. 7B in the case of a vertebra where it may be advantageous not to apply pressure towards the proximal part which could, for example, be situated near a wall of bone tissue. In the example of FIG. 7B, the implant is deployed by applying a pushing force causing the flanges to pivot towards the distal end, and the posterior wall of the vertebral body is thus preserved and the risks of breakage are limited.

FIGS. 8A, 8B and 8C depict, respectively, a view from above, a face-on view of the profile view of an expandable implant with three flanges according to certain embodiments. FIGS. 9A and 9B depict perspective views respectively from the rear and from the front of an expandable implant according to the embodiments of FIGS. 8A, 8B and 8C. FIGS. 8A, 8B, 8C, 9A and 9B depict embodiments in which the implant comprises more than two flanges and particularly in these examples a third flange, but it is clear that the invention may also cover four, or even more, flanges, although in general three or four flanges will suffice because three-dimensional deployment will generally be satisfactory with such configurations. In these embodiments, it is clear that deployment of the implant makes it possible to restore an additional number of bony structures compared with implants comprising just two flanges, particularly long bones. The deployment of such types of implant provides a better guarantee of the expansion volume obtained compared with that desired, whether or not the implant is supplemented by the injection of cement (and particularly whether or not it has a casing providing containment of the volume of injected cement). In addition, as mentioned hereinabove, the use of support arms of different sizes makes it possible to obtain flanges with various shapes once deployed, and for example a concave upper flange and two planar lateral flanges that are either straight or inclined, or any combination between these straight-planar, inclined-planar or concave or convex configurations.

FIGS. 10A, 10B and 10C depict profile views of vertebrae that have respectively suffered anterior, median and posterior vertebral compression fractures (VCFs) . The invention allows these kind of vertebral fracture to be treated by arranging the deployable implant in the correct position in the plane of the implantation site, using antero-posterior and/or medio-lateral positioning and adjusting the depth of insertion and/or the angle of insertion of the implant, according to the type of surgical approach being used (for example lateral, anterior, dorsal, transforaminal, transpedicular, etc.) .

FIG. 12A depicts a perspective view of an implant according to certain embodiments and FIGS. 12B and 12C depict, respectively, the detail of the boxes 12B and 12C of FIG. 12A of the implant. In these embodiments, the implant has a length suitable for restoring the geometry of bone structures of long length, for example such as long bones. The example depicted in these FIG. 12 only comprises two flanges, but it is obvious that it could comprise three or four of them, and the length of the implant is designed to allow, for example, intramedullary insertion into a long bone. In such embodiments, the expansion arms will be variable in number and will generally be present only at the ends of the central access and of the implant, since expansion is generally desired near to this location, generally intended to be placed at the epiphyses. However, it is of course possible to have support arms that are distributed anywhere along the longitudinal axis of the implant. In the case of intramedullary implantation, the use of support arms only at the ends of the implant makes it possible to conform to the internal shape of the cavity, which is generally flared at the epiphyseal ends. In addition, notches (133, 143) provided on the flanges at various points (generally intended to be placed near the epiphyses) allow bowing of the flanges while offering better flexibility locally. In addition, it is possible to provide a number of support arms that differs between the proximal end and the distal end according to the type of expansion desired. For example, in the case of long bones ending in a head which generally comprises a medullary cavity of greater diameter than the other end of the long bone, it is possible to provide implants of different lengths that are suited to the different types of bone such as the humerus, the femur, etc. with suitable diameters, a suitable number of flanges and an expansion providing a final deployed shape that varies according to the type of bone and the desired geometric restoration.

FIG. 11A depicts a perspective view of an expandable implant in the deployed configuration and equipped with a lock that holds the implant in its deployed configuration, FIG. 11B depicts a detail of the proximal end of an implant of the type of FIG. 11A, with the lock on the outside of the implant according to certain embodiments, and FIG. 11C depicts a detail of the proximal end of an implant according to certain embodiments with another type of lock on the outside of the implant. In particular, FIGS. 11A and 11B depict embodiments in which the implants, once deployed, can be locked by a screw lock (VV). In such embodiments, the proximal end (11) has a tapped threaded conduit into which there may be introduced a screw lock (VV) that has a screw thread on its periphery to collaborate with this thread. Tightening the screw lock (VV) applies pressure to the central shaft (3) to prevent it from moving towards the proximal end and causing the re-folding of the implant which thus finds itself locked in the deployed position. Such a lock is preferably provided with a central bore and possibly with distal holes to allow cement to be injected into the implant, as in certain embodiments already described hereinabove. Moreover, in order to tighten this lock (VV), its proximal end is fitted with means for collaborating with a screw-turning tool, such as for example peripheral wings depicted in FIGS. 11A and 11B. By contrast, in FIG. 11C, the locking mechanism relies on teeth. This mechanism comprises a toothed lock (VC) which contains a circumferential groove able to accept a split ring acting as a locking clip intended to collaborate with a circumferential housing inside the conduit into which the toothed lock is inserted to form this clip. As depicted in FIG. 11C, as the toothed lock is pushed in, the split ring engages with teeth in the conduit of the implant which can thus be locked incrementally by this lock pressing against interior teeth in the conduit of the implant. These teeth are preferably asymmetrical so as to allow the lock to be withdrawn only towards the outlet, thereby keeping the implant secure in the deployed position and preventing it from re-folding under the force exerted by the surrounding tissue.

The present application describes various technical and advantageous features with reference to the figures and/or to various embodiments. The person skilled in the art will appreciate that the technical features of one given embodiment may in fact be combined with features of another embodiment unless the contrary is explicitly mentioned or unless it is obvious that these features are incompatible or that combining them will not provide a solution to at least one of the technical problems mentioned in the present application. In addition, the technical features described in one given embodiment may be taken in isolation from the other features of this embodiment unless the contrary is explicitly mentioned.

Detailed list of references in the figures:
1 implant
11 proximal end
12 distal end
A implantation instrument
Ac fluid-injection instrument
A1 hollow holding tube
A3 expansion rod?
3 central shaft
31 central-shaft conduit
32 openings in the central-shaft conduit
13 first flange
14 second flange
15 third flange
20 expansion ring
131 first-flange support arm
141 second-flange support arm
151 third-flange support arm
132 first-flange expansion arm
133 notch on the first tray
142 second-flange expansion arm
143 notch on the second flange
152 third-flange expansion arm
130 first-flange central support arm
140 second-flange central support arm
150 third-flange central support arm
VC toothed lock
VV screw lock

1. An expandable bone implant (1) for human orthopaedic surgery for restoring the volume and/or geometry of a bone by expansion between a folded configuration and a deployed configuration, said implant comprising a central shaft (3) and extending along a longitudinal axis (L) between a proximal end (11) adapted to cooperate with an implantation instrument (A) for holding the implant and a distal end (12) intended to be first inserted into the bone, at least two faces of the implant each comprising at least one flange (13, 14, 15) for contact with bone tissue, each of the flanges being supported by at least two support arms (131, 141, 151) each, by means of a hinge on the central shaft (3) and a hinge under the respective flange (13, 14, 15) of each of said support arms (131, 141, 151);
wherein:
an expansion ring or socket (20) is arranged on the same axis as said central shaft (3);
at least two expansion arms (132, 142, 152) each have a hinge connecting them to one of the ends of one of the flanges (13, 14, 15) and a hinge connecting them to said expansion ring or socket (20);
said expansion ring or socket (20) and the proximal end of the central shaft (3) are able to be actuated in order to cause separation between said socket (3) and said central shaft (3), applying traction to the flanges (13, 14, 15), by means of the expansion arms (132, 142, 152), thereby causing said support arms (131, 141, 151) to pivot, causing the flanges (13, 14, 15) to move away from the central shaft (3), so as to result in controlled expansion of the implant (1) between said folded configuration and said deployed configuration.

2. The implant according to claim 1, wherein it is said expansion ring (20) that is operable by being able to collaborate, by means of attachment, with a hollow tube (A1) used by said implantation instrument (A) for holding the implant (1), letting the expansion rod (A3) pass through the ring, while the central shaft (3) is able to collaborate with said expansion rod (A3) of said instrument (A) so that a pressing force exerted on said expansion rod (A3) sliding inside said hollow tube (A1) causes said central shaft (3) to move away from said ring (20), resulting in controlled expansion of the implant (1) dependent on the pressing force exerted on the expansion rod (A3).

3. The implant according to claim 1, wherein it is the proximal end of the central shaft (3) that is able to collaborate with said hollow tube (A1) used by said implantation instrument (A) for holding the implant (1), whereas said expansion ring or socket (20) is able to collaborate with said expansion rod (A3) of said instrument (A) so that a pressing force exerted on said expansion rod (A3) sliding inside said hollow tube (A1) causes said expansion ring or socket (20) to move away from said central shaft (3), resulting in controlled expansion of the implant (1) dependent on the pressing force exerted on the expansion rod (A3).

4. The implant according to claim 1, wherein the flanges are provided, on their surface of contact with the bone tissue, with irregularities of shape such as protuberances or, conversely, invaginations to improve the gripping of the flanges on the bone tissue.

5. The implant according to claim 1, wherein the central shaft (3) comprises a conduit (31) able to collaborate with said expansion rod (A3) which is, on the one hand, hollow and provided with at least one opening at its distal end and, on the other hand, able to be connected to a fluid-injection instrument (AC) for conveying at least one fluid to the implant implantation site via the central shaft (3), and which is preferably provided with openings (32) to disgorge the fluid along the length of the central shaft (3).

6. The implant according to claim 1, wherein the flange-support arms (131, 141, 151) comprise two arms connected respectively near the proximal and distal ends of their respective flange (13, 14, 15) to provide support along the entire length of the flanges and limit the risk of them bowing or sagging.

7. The implant according to claim 1, wherein at least one additional central support arm (130, 140, 150) is connected between a central portion of the central shaft and a central portion of the flanges (13, 14, 15), with hinges at the two ends of the support arm (130, 140, 150) to allow it to pivot.

8. The implant according to claim 1, wherein the distal-end support arms have a length different from that of the proximal-end support arms and of the central support arms.

9. The implant according to claim 1, wherein it comprises two flanges (13, 14) arranged one on each side of the central shaft (3) to provide a bearing surface to bear against the damaged bone tissue on each side of the implant.

10. The implant according to claim 1, wherein it comprises at least one additional flange (15), the distribution of the flanges about the central shaft (3) varying according to the number of them and/or according to what is needed in terms of surgical intervention, these preferably being equally angularly distributed radially with respect to the central shaft so as to apply uniform compression to the bone tissue at the periphery of the implant.

11. A system for orthopaedic treatment of damaged bone tissue, comprising a bone substitute cement and at least one instrument (A) for implantation and for injection (Ac) of cement into the implant (1), wherein it comprises the implant (1) according to claim 1.

12. The system according to claim 11, wherein the instrument (A) for implantation and for injection (Ac) of cement comprises means for controlling the pressure and/or the aspiration of the cement so that the implant can be re-folded to the folded configuration if necessary.

13. The system according to claim 11, wherein the implantation instrument (A) is distinct from but complements the injection instrument (Ac) the cement-injection canal of which passes through a canal inside the rod of the implantation instrument (A) that via its distal end holds the proximal end of the implant (1) .
